(19) United States
(12) Patent Application Publication
(10) Pub. No.: US 2012/0309997 A1
(43) Pub. Date: Dec. 6, 2012

(54) Enantiomerically Enriched Aminodiphosphines as Ligands for the Preparation of Catalysts for Asymmetric Synthesis

(75) Inventors: Mónica Alonso Xalma, Barcelona (ES); Xavier Verdaguer Espaulella, Vic (ES); Marc Revés Vilaplana, Barcelona (ES); Antoni Riera Escalé, Barcelona (ES)

(73) Assignee: ENANTIA, S.L., Barcelona (ES)

(21) Appl. No.: 13/578,247

(22) PCT Filed: Oct. 13, 2010
(86) PCT No.: PCT/EP2010/065366

§ 371 Date: Aug. 9, 2012

(30) Foreign Application Priority Data
Feb. 12, 2010 (EP) 10382034.6

(51) Int. Cl. C07F 17/00; C07C 233/65; C07F 5/02

(52) U.S. Cl. 556/7; 564/8; 560/43

(57) ABSTRACT

The present invention relates to enantiomerically enriched aminodiphosphine ligands where the chirality is located in the phosphorus atom and their preparation process, to catalysts containing them and their preparation process, as well as their use in asymmetric synthesis.

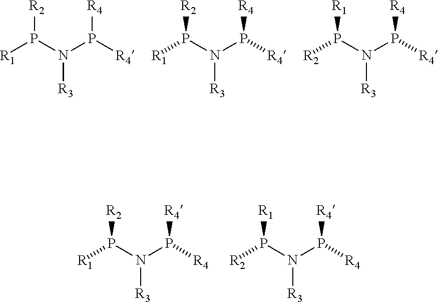

The present invention relates to an enantiomerically enriched aminodiphosphine ligands and their preparation process, to catalysts containing them and their preparation process, as well as their use in asymmetric synthesis.

### BACKGROUND ART

Phosphorus atom surrounded by three different substituents can be configurally stable leading a pair of enantiomeric forms. The application of substances possessing these chiral phosphorus atoms as ligands for the preparation of catalysts for asymmetric synthesis has promoted active research. Thus, chiral catalysts containing the chiral phosphorus atom ligands mentioned above have been used in some representative catalytic asymmetric reactions such as hydrogenation, hydrosilylation of ketones and conjugate addition of Michael reaction. (cf. Arnald Grabulosa et al. “Preparation of optically pure P-stereogenic trivalent phosphorus compounds”, Coordination Chemistry Reviews, 2007, Vol. 251, pp. 25-90; and Yoshinori Yamanoi et al. “Methylene-bridged P-chiral diphosphines in highly enantioselective reactions”, Journal of Organic Chemistry, 1999, Vol. 64, pp. 2988-2989).

In particular, catalytic asymmetric hydrogenation is one of the most powerful tools for the synthesis of enantiomerically pure compounds which can have a profound impact in obtaining intermediates for the production of high value-added products such as pharmaceuticals or fine chemicals. Chemists have developed many approaches for obtaining enantiomerically pure compounds by asymmetric hydrogenation using synthetic chiral catalysts. During the last decade, great attention has been devoted to discover new asymmetric catalysts as a key in the production of enantiomerically pure compounds.

Certain chiral diphosphine ligands have been successfully used to mediate catalytic asymmetric hydrogenation. In particular, high enantioselectivity has been achieved using the C₁-symmetrical chiral diphosphine ligand of formula (methyl)(tert-butyl)PCH₂P(tert-butyl)₂ for the preparation of rhodium complexes useful as catalysts for the asymmetric hydrogenation of alpha-acetamido dehydroamino acids. Each enantiomer of the above-mentioned C1-symmetrical chiral diphosphine ligand has been obtained separating a diborane complex thereof by chiral preparative high performance liquid chromatography (preparative HPLC).

Complexes such as rhodium complexes can be prepared by reaction of these ligands with [Rh(COD)₂]⁺ BF₄⁻. In general, the chiral phosphorus-carbon-phosphorus (PCP) ligands are oils sensitive to oxidation after air exposure. Therefore, it is convenient to transformate immediately into the metal complex and use the catalysts obtained within a maximum period of few hours (cf. Garret Hoge et al. “Highly selective asymmetric hydrogenation using a three hindered quadrant bisphosphine rhodium catalyst”, Journal of the American Chemical Society, 2004, Vol. 126, pp. 5966-5967; Ilya D. Gridnev et al. “asymmetric hydrogenation catalyzed by a rhodium complex of (R)-(tert-butylmethylphosphino)(di-tert-butylphosphino)methane: Scope of Enantioselectivity and Mechanistic Study”, Journal of the American Chemical Society, 2008, Vol. 130, pp. 2560-2572; and the international patent application number WO 2005/87370).

Bis(diarylphosphino)amines of general formula Ar₁Ar₂P—N(R₂)—PAr₃Ar₄ are known compounds used as ligands olefin oligomerization catalyst (see for instance documents WO 01/10876, WO 2008/077908, US 2007/027350, WO 2004/056480, WO 2004/056479, WO 02/04119, WO 2010/034101, Overett et al: Chem. Commun., 2005, pp. 622-624, and Du Toit, Aletta et al: “Styrene-ethylene Co-oligomerization with bis-(diphenylphosphino)-amine/chromium catalysts and the use of the co-oligomerization products in copolymerization reactions with metallocenes “Journal of polymer science, Part A: Polymer chemistry, 2008, vol. 46(4), pp. 1488-1501). Even a more general family of compounds of general formula (R¹)₂P—X—P(R¹)ₘ(R²)ₙ has been also claimed as ligands in catalytic oligomerization processes (see WO 2007/057458). In all these references, however, most of the ligands are achiral and the few cases of chiral compounds are described only as mixtures of racemate and meso isomers. Therefore, no single non racemic bis(diarylphosphino)amine has been prepared in those references.

Two bis(dialkylphosphino)amines have been described to date (see Cowley, A. H. et al: “Nitrogen-15-proton coupling constant study of the bonding in some nitrogen-phosphorus, nitrogen-arsenic, nitrogen-sulfur, and nitrogen-silicon compounds”, Journal of the American Chemical Society, 1973, vol. 95(13), pp. 4179-85, and Burg et al: J. Am. Chem. Soc., 1966, vol. 88, pp. 31-37). These chiral phosphines, bearing CH₃CF₃P groups, were prepared as an inseparable mixture of meso and racemic isomers.

Only one chiral non racemic P—N—P ligand has been described (see Venkatakrishnan et al: “Ruthenium hydride complexes of chiral and achiral diphosphazane ligands and asymmetric transfer hydrogenation reactions” Journal of Organometallic Chemistry, 2007, vol. 692, pp. 1875-1891; Mandal, Swadhin K. et al: “Palladium(II) allyl complexes of chiral diphosphazane ligands: Ambident coordination behaviour and stereodynamic studies in solution” Dalton Transactions, 2003, pp. 1016-1027; and Venkatakrishnan, Thengarai S. et al: “Ruthenium carbonyl clusters derived from pyrazolyl substituted diphosphazanes: crystal and molecular structure of a triruthenium cluster featuring a triply bridging μ₃-η¹:η¹:η¹ coordination mode of pyrazolate moiety” Journal of Organometallic Chemistry, 2006, vol. 691 (1-2), pp. 224-228). (S_{C}R_{P})-Ph₂PN(R)PPh(N₂—C₃HMe₂-3,5) [R=*CHMePh] is a pyrazolyl diphosphazane that has been used in the preparation of several organometallic complexes and screened in asymmetric synthesis. However, this ligand was not capable to give any asymmetric bias to any of the reactions that were tested. We can conclude that as a ligand in asymmetric synthesis this compound is completely useless. Perhaps due to the presence of the pyrazolyl substituent or because it is not sterically encumbered, this phosphazane gave only low levels of enantiomeric excess either in transfer hydrogenation or in allylic alkylation.

Several chiral aminomonophosphines of general structure RR′PNHCHR″R′″ where R and R′ can be a C₁-C₄ alkyl or a phenyl, R″ is a C₁-C₄ alkyl, and R′″ can be a phenyl or a COOCH₃, and their oxide or thiooxide have been synthesized by the reaction of the corresponding borane complex of the aminomonophosphine with a base. In these compounds the chirality is located in the phosphorus atom. Chemists are silent of the potential use of these chiral aminomonophosphines in the preparation of enantiomerically enriched aminodiphosphines of the general formula phosphorus-nitrogen-phosphorus (PNP) (cf. E. V. Grishkun et al. “synthesis of chiral tert-butyl-phenylphosphine oxide”, Russian Journal of General Chemistry, 2003, Vol. 73, pp. 1823-1824; Oleg I. Kolodiazhnyi et al, “Asymmetric synthesis of chiral N-(1-methylbenzyl)aminophosphines”, Tetrahedron: Asymmetry, 2003, Vol. 14, pp. 181-183; O. I. Kolodyazhnyi et al., “Stereoselective Reactions of Optically Active Derivatives of alpha-methylbenzylaminophosphine”, Russian Journal of General Chemistry, 2004, Vol. 74, pp. 515-522; Evgenyi V. Gryshkun et al. “Stereoselective reactions of chiral amines with racemic chlorophosphines”, Phosphorus, sulfur, and silicon and the related elements), 2004, Vol. 179, pp. 1027-1046).

Thus, the research of chiral aminodiphosphine ligands PNP for manufacturing metal complexes useful as catalysts for asymmetric hydrogenation reactions is still an active field since the known chiral diphosphine ligands of general formula PCP have the disadvantages of their instability, requiring their fast transformation into the metal complexes. In addition, the known diphosphine ligands PCP are obtained by tedious processes difficult to carry out at industrial scale since the use of chromatographic techniques is required.

Therefore, from what is known in the art it is derived that there is still the need of providing stable and easy-prepared enantiomerically enriched aminodiphosphine ligands for preparing catalysts to be used in asymmetric reactions, in particular, in asymmetric hydrogenation reactions.

### SUMMARY OF THE INVENTION

Inventors have found that an enantiomerically enriched (i.e. chiral, non-racemic) ligand of general structure P—N—P where the chirality is located at least in one of the phosphorus atoms is useful for the preparation of catalysts for asymmetric synthesis, in particular asymmetric hydrogenation reactions of amino acids. The use of these catalysts allows obtaining high enantioselectivity and purity of the obtained compounds. The preparation of the ligands of the present invention based on chiral aminophosphines allows achieving higher enantiomerically enriched ligands by crystallization avoiding the use of chromatographic techniques. It is also advantageous because intermediates for the preparation of the ligands of the present invention have also been found to be stable.

Thus, an aspect of the present invention relates to an enantiomerically enriched ligand of formula (I),

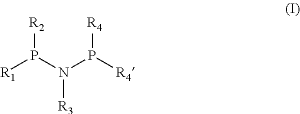

or any of its stereoisomers which are:

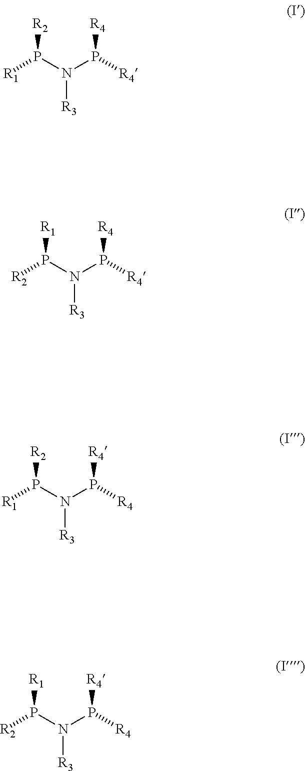

or their salts in any of their tautomeric forms, or their borane complexes in any of their tautomeric forms, where:

R₁, R₂, R₄ and R₄′ are radicals independently selected from the group consisting of C₁-C₄ alkyl unsubstituted or substituted with one or more groups Rₐ, phenyl C₁-C₄ alkyl unsubstituted or substituted with one or more groups Rₐ, C₂-C₄ alkenyl unsubstituted or substituted with one or more groups Rₐ, a 5 to 6 membered carbocyclic monocyclic ring unsubstituted or substituted with one or more groups Rₐ, a 6 to 12 membered bridged carbocyclic polycyclic ring unsubstituted or substituted with one or more groups Rₐ, and a 8 to 12 membered fused carbocyclic polycyclic ring unsubstituted or substituted with one or more groups Rₐ, being the ring saturated, partially unsaturated or aromatic; or alternatively R₄ and R₄′ form, together with the P atom to which they are bound, a 5 to 12 known membered monocyclic, bicyclic, bridged or fused polycyclic ring, being the ring saturated, partially unsaturated or aromatic; the members of the ring being independently selected from C, N, O, S, and P; each Rₐ is independently selected from the group consisting of Cᵣ C₄ alkyl, halo C₁-C₄ alkyl, halogen, C₁-C₄ alkoxy, halo C₁-C₄ alkoxy, C₁-C₄ alkylthio, and CN; R₃ is hydrogen or C₁-C₄ alkyl; wherein R₁, R₂ are different radicals; and if R₄ and R₄′ are different, R₁ is equal to R₄, and R₂ is equal to R₄′, then the chirality of the phosphorus atoms is not RS or SR.

Another aspect of the present invention relates to a compound which comprises an enantiomerically enriched ligand of formula (I) or any of its stereoisomers as defined above, and a metal complex of formula [M^{a+}(L₁)ₘ(L₂)ₙ](A^{b-}), the metal of the metal complex being bound to the ligand through the phosphorus atoms, wherein: M is a metal selected from the group consisting of Ru, Rh, Ir, and Cu; L₁ is a diene selected from the group consisting of 1,5-cyclooctadiene, norbornadiene, and 2,5-dimethyl-hexa-1,5-diene; L₂ is an anionic ligand selected from the group consisting of Cl⁻, Br⁻, I⁻, ⁻CN, OR₁₆, and ⁻R₁₆ or a neutral σ-donor ligand selected from the group consisting of NR₁₆R₁₇R₁₈, R₁₆OR₁₇, R₁₆SR₁₇, CO, and NCR₁₆; R₁₆, R₁₇ and R₁₈ are independently selected from the group consisting of hydrogen and C₁-C₆ alkyl; A is an anion selected from the group consisting of OTf⁻, PF₆⁻, BF₄⁻, SbF₆⁻, and ClO₄⁻; m is an integer from 0 to 3, inclusive; n is an integer from 0 to 3, inclusive; m+n is an integer from 0 to 3 inclusive; b is the number of negative charges of the anion; and a is the number of positive charges of the metal ion.

Another aspect of the present invention relates to a process for the preparation of the enantiomerically enriched ligands of formula (I) or any of its stereoisomers, or their salts in any of their tautomeric forms, or their borane complexes in any of their tautomeric forms as defined above, which comprises:

(a) reacting an enantiomerically enriched compound of formula (III) or alternatively of formula (III′), in the presence of a strong base,

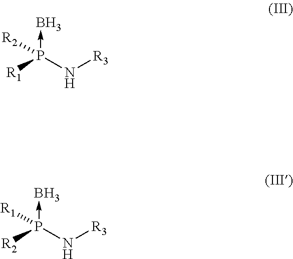

with a compound of formula (IV),

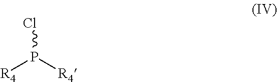

where R₁, R₂, R₃, R₄ and R₄′ are as defined above and the symbol [symbol] means any of the two possible configurations of the phosphorus atom attached to the chlorine atom; (b) reacting the aminodiphosphine borane complex obtained in step (a) with a base or an acid; (c) isolating the compound of formula (I) or any of its stereoisomers in form of free base or as a salt; and (d) optionally, converting the free base of step (c) into a salt by reaction with the corresponding acid or converting the salt of step (c) into the free base by reaction with a base.

Another aspect of the present invention relates to a process for the preparation of an enantiomerically enriched compound of formula (III) or alternatively of formula (III′), wherein R₁, R₂, and R₃ are as defined above, which comprises:

(i) reacting an enantiomerically enriched compound of formula (V) or alternatively of formula (V′),

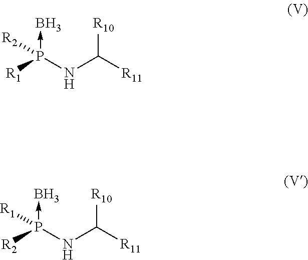

with a solution of an alkaline metal selected from L₁ and Na, and ammonia; or alternatively reacting with hydrogen or a hydrogen source in the presence of a metal catalyst selected from the group consisting of Pd, Pd on carbon and Pd(OH)₂; to yield a compound of formula (III) or alternatively of formula (III′) where R₃ is hydrogen; or

(ii) (a) reacting an enantiomerically enriched compound of formula (V) or alternatively of formula (V′) as defined above with R₃X in the presence of a strong base; and (b) reacting the resulting compound of step (a) with a solution of an alkaline metal selected from Li and Na, and ammonia; or alternatively reacting with hydrogen or a hydrogen source in the presence of a metal catalyst selected from the group consisting of Pd, Pd on carbon and Pd(OH)₂; to yield a compound of formula (III) or alternatively of formula (III′) where R₃ is C₁-C₄ alkyl.

In the previous formulas, R₁₀ and R₁₁ are different radicals independently selected from the group consisting C₁-C₄ alkyl unsubstituted or substituted with one or more groups Rₐ, phenyl C₁-C₄ alkyl unsubstituted or substituted with one or more groups Rₐ, C₂-C₄ alkenyl unsubstituted or substituted with one or more groups Rₐ, COR₁₂, NCOR₁₅, a 5 to 6 membered monocyclic ring unsubstituted or substituted with one or more groups Rₐ, a 6 to 12 membered bridged polycyclic ring unsubstituted or substituted with one or more groups Rₐ, and a 8 to 12 membered fused polycyclic ring unsubstituted or substituted with one or more groups Rₐ, being the ring saturated, partially unsaturated or aromatic; R₁₂ is selected from the group consisting of C₁-C₄ alkyl unsubstituted or substituted with one or more groups Rₐ, C₁-C₄ alkoxy, and NR₁₃R₁₄; R₁₃ and R₁₄ are independently selected from the group consisting of hydrogen, and C₁-C₄ alkyl unsubstituted or substituted with one or more groups Rₐ; R₁₅ is selected from the group consisting of C₁-C₄ alkyl unsubstituted or substituted with one or more groups Rₐ, C₁-C₄ alkoxy, and NR₁₃R₁₄; each Rₐ is independently selected from the group consisting of C₁-C₄ alkyl, phenyl C₁-C₄ alkyl, halo C₁-C₄ alkyl, halogen, C₁-C₄ alkoxy, halo C₁-C₄ alkoxy, C₁-C₄ alkylthio and CN; and X is halogen selected from the group consisting of chloro, bromo, and iodo.

Another aspect of the present invention relates to a process for the preparation of the compound which comprises an enantiomerically enriched ligand of formula (I) or any of its stereoisomers as defined above, and a metal complex of formula [M^{a+}(L₁)ₘ(L₂)ₙ](A^{b-}), the metal of the metal complex being bound to the ligand through the phosphorus atoms, which comprises reacting an enantiomerically enriched ligand of formula (I) or any of its stereoisomers, with a metal complex of formula [M^{a+}(L₁)ₘ(L₂)ₙ](A^{b-}), where M, L₁, L₂, A, m, n, m+n, b, and a are as defined above.

Another aspect of the present invention relates to the use of the previous compound as catalyst for asymmetric reactions.

Another aspect of the present invention relates to a process for performing asymmetric hydrogenation reactions which comprises reacting a prochiral or chiral compound in the presence of the catalyst of the invention under pressure with hydrogen or a hydrogen source, to produce an optically active compound.

Another aspect of the present invention relates to an enantiomerically enriched intermediate compounds of formula (III) or alternatively of formula (III′), or their salts,

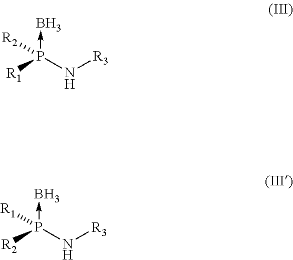

where R₁, R₂, and R₃ are as defined above.

Finally, another aspect of the present invention relates to the enantiomerically enriched compounds of formula (V) or formula (V′), or their salts, where R₁, R₂, R₁₀ and R₁₁ are as defined above; with the proviso that compounds of formula (V) or (V′) is not a compound of the following list:

Borane complex of (S)—P-tert-butyl-P-phenyl-N—[(S)-1-phenylethyl] phosphinamine (V; R₁=phenyl, R₂=tert-butyl, R₁₀=methyl, R₁₁=phenyl);

Borane complex of (R)—P-iso-butyl-P-tert-butyl-N—[(S)-1-phenylethyl] phosphinamine (V; R₁=iso-butyl, R₂=tert-butyl, R₁₀=methyl, R₁₁=phenyl);

Borane complex of (S)—P-phenyl-P-trimethylphenyl-N—[(S)-1-phenylethyl] phosphinamine (V; R₁=phenyl, R₂=2,4,6-trimethylphenyl, R₁₀=methyl, R₁₁=phenyl); and

Borane complex of (S)—P-tert-butyl-P-phenyl-N-[(1-carbomethoxy-2-methyl)propyl] phosphinamine (V; R₁=phenyl, R₂=tert-butyl, R₁₀=iso-butyl, R₁₁=COOCH₃).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

In the context of the invention, the term “enantioselectivity” refers to a given reaction (e.g. hydrogenation) that yields more of one enantiomer than another.

The “enantiomerically excess” or “ee” is a measure of the excess of one enantiomer over a racemic mixture of a chiral compound, which is commonly expressed as a percentage. Enantiomeric excess is defined as the absolute difference between the mole fraction of each enantiomer [ee ═F(+)−F(−)]. If the moles of each enantiomer are known, the percent enantiomeric excess can be determined by the following formula: ee=((R−S)/(R+S))×100, where R and S are the respective fractions of enantiomers in the mixture such that R+S=1.

The term “enantiomerically enriched” refers to a chiral non-racemic compound, that is, a compound which has more of one enantiomer than another. The degree of enrichment is measured by the ee. Therefore, those chiral compounds which are racemic (i.e., which comprise a mixture of enantiomers any of them in the same amount) or meso isomers (i.e. which comprise an internal plane of symmetry), or mixtures thereof do not form part of the present invention.

In the formula of the compounds of the present invention, the use of bold and dashed lines to denote particular configuration of groups follows the IUPAC convention. A bond indicated by a broken line indicates that the group in question is below the general plane of the molecule as drawn (the “alpha” configuration), and a bond indicated by a bold line indicates that the group at the position in question is above the general plane of the molecule as drawn (the “beta” configuration).

The term C₁-C₄ alkyl refers to a saturated branched or linear alkyl chain which contains from 1 to 4 carbon atoms. Examples include the group methyl, ethyl, propyl, isopropyl, butyl, isobutyl, sec-butyl, and tert-butyl.

The term C₂-C₄ alkenyl refers to a branched or linear alkyl chain which contains from 2 to 4 carbon atoms and that also contains one or two double bonds. Examples include, among others, ethenyl, 1-propen-1-yl, 1-propen-2-yl, 3-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, and 1,3-butadien-2-yl.

The term halo C₁-C₄ alkyl refers to a group resulting from the replacement of one or more hydrogen atoms from a C₁-C₄ alkyl group with one or more halogen atoms (i.e. fluoro, chloro, bromo or iodo), which can be the same or different. Examples include, among others, trifluoromethyl, fluoromethyl, 1-chloro ethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl, and nonafluorobutyl.

A halogen radical means fluoro, chloro, bromo or iodo.

The term C₁-C₄ alkoxy refers to an alkoxy group having from 1 to 4 carbon atoms, the alkyl moiety having the same meaning as previously defined. Examples include methoxy, ethoxy, propoxy, isopropoxy, butoxy, isobutoxy, sec-butoxy, and tert-butoxy.

The term phenyl C₁-C₄ alkyl refers to a group resulting from the replacement of one or more hydrogen atoms from a C₁-C₄ alkyl group with one or more phenyl groups. Examples include benzyl or phenylethyl.

The term halo C₁-C₄ alkoxy refers to a group resulting of the replacement of one or more hydrogen atoms from a C₁-C₄ alkoxy group with one or more halogen atoms (i.e. fluoro, chloro, bromo or iodo), which can be the same or different. Examples include, among others, trifluoromethoxy, fluoromethoxy, 1-chloroethoxy, 2-chloroethoxy, 1-fluoroethoxy, pentafluoroethoxy, 3-fluoropropoxy, 3-chloropropoxy, 2,2,3,3-tetrafluoropropoxy, 2,2,3,3,3-pentafluoropropoxy, heptafluoropropoxy, 4-fluorobutoxy, and nonafluorobutoxy.

The term C₁-C₄ alkylthio refers to a branched or linear alkyl chain which contains from 1 to 4 carbon atoms, the alkyl moiety having the same meaning as previously defined. Examples include methylthio, propylthio, isopropylthio, butylthio, isobutylthio, sec-butylthio, and tert-butylthio.

The term bridged or fused “polycyclic” ring refers to a ring system which contains from 2 to 4 rings. The rings can be saturated, partially unsaturated or aromatic, which may be substituted or unsubstituted as described herein.

The term “carbocyclic” ring refers to a ring system wherein all the ring members are C. The term “heterocyclic” ring refers to a ring system wherein one or more of the ring members, preferably 1, 2, 3, 4 ring members, is selected from N, O, S, and P. Both the carbocyclic and heterocyclic rings can be saturated, partially unsaturated or aromatic, and may be substituted or unsubstituted as described herein.

The term “known ring system” refers to a ring system which is known in the art and so intends to exclude those ring systems that are not chemically possible.

The expression “substituted with one or more” means that a group can be substituted with one or more, preferably with 1, 2, 3 or 4 substituents, provided that this group has 1, 2, 3 or 4 positions susceptible of being substituted.

As mentioned above, an aspect of the present invention refers to an enantiomerically enriched ligand of formula (I) or any of its stereoisomers, or a salt thereof in any of its tautomeric forms or a borane complex thereof in any of its tautomeric forms useful for the preparation of a catalyst.

Compounds of formula (I) or any of its stereoisomers can be in form of salts of either organic or inorganic acids. Examples of appropriate inorganic acids include hydrochloric acid, hydrobromic acid, hydroiodic acid, nitric acid, perchloric acid, sulfuric acid or phosphoric acid. Examples of appropriate organic acids include methansulfonic acid, trifluoromethansulfonic acid, ethansulfonic acid, benzenesulfonic acid, p-toluenesulfonic acid, fumaric acid, citric acid, oxalic acid, acetic acid and maleic acid, among others. A preferred salt of the ligands of formula (I) or any of its stereoisomers is the hydrochloride salt. Another preferred salt of the ligands of formula (I) any of its stereoisomers is the tetrafluoroborate salt.

Optionally, the compounds of formula (I) or any of its stereoisomers can be in form of a borane complex such as BH₃ borane complex.

As previously mentioned the salts and borane complexes of the compounds of formula (I) can be in any of their tautomeric forms. Thus, for example a monosalt of the compound of formula (I) wherein R₃ is hydrogen may be in the following tautomeric form:

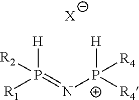

or in any of its stereoisomeric forms, wherein R₁, R₂, R₄ and R₄′ are as previously defined and X is the salt anion.

In comparison with the oily chiral PCP diphosphine ligands known in the art, the enantiomerically enriched aminodiphosphine ligands PNP of formula (I) any of its stereoisomers can be in form of salts, which have shown to be solid and stable compounds against oxidation via air exposure. Their stability allows their easy handling and the increasing of the storage period, avoiding the need to be rapidly transformed into the chiral catalyst.

In a preferred embodiment ligands of formula (I) or any of its stereoisomers have an ee. equal or greater than 95%. In another preferred embodiment ligands of formula (I) any of its stereoisomers have an ee. equal or greater than 98%. In a still more preferred embodiment, ligands of formula (I) any of its stereoisomers have an ee. equal or greater than 99%.

In a particular embodiment, ligands of formula (I) or any of its stereoisomers are those where R₄ and R₄′ are different, wherein if R₁ is equal to R₄, and R₂ is equal to R₄′, then the chirality of the phosphorus atoms is RR or SS.

In a preferred embodiment ligands of formula (I) or any of its stereoisomers are those where: R₁ and R₄ are C₁-₄ alkyl; R₃ is hydrogen, and R₂ and R₄′ are C₁-₄ alkyl or substituted or unsubstituted phenyl.

Examples of these ligands are selected from the following list:

(P_{S})(P_{S})-di(tert-butylmethylphosphino)amine (R₁=methyl, R₂=tert-butyl, R₃=hydrogen, R₄=methyl, R₄′=tert-butyl);

(P_{R})(P_{R})-di(tert-butylmethylphosphino)amine (R₁=methyl, R₂=tert-butyl, R₃=hydrogen, R₄=methyl, R₄′=tert-butyl);

(P_{S})(P_{S})-di(methylphenylphosphino)amine (R₁=methyl, R₂=phenyl, R₃=hydrogen, R₄=methyl, R₄′=phenyl);

(P_{R})(P_{R})-di(methylphenylphosphino)amine (R₁=methyl, R₂=phenyl, R₃=hydrogen, R₄=methyl, R₄′=phenyl); and

(P_{S})(P_{S})—N,N′-bis(tert-butylmethylphosphino)methylamine (R₁=tert-butyl, R₂=methyl, R₃=methyl, R₄=methyl, R₄′=tert-butyl).

In a preferred embodiment, the ligands of formula (I) or any of its stereoisomers are those where: R₁, R₂, R₄ and R₄′ are radicals independently selected from the group consisting of substituted or unsubstituted C₁-C₄ alkyl, a substituted or unsubstituted 5 to 6 membered carbocyclic monocyclic ring, and a substituted or unsubstituted 6 to 12 membered bridged carbocyclic polycyclic ring. In a more preferred embodiment, the 5 to 6 membered carbocyclic monocyclic ring is selected from phenyl and cyclohexyl, and the 6 to 12 membered bridged carbocyclic polycyclic ring is adamantyl. In a still more preferred embodiment, the C₁-C₄ alkyl, the cyclohexyl, and the adamantyl are unsubstituted. In a still more preferred embodiment, the C₁-C₄ alkyl is methyl or tert-butyl.

In another preferred embodiment, ligands of formula (I) or any of its stereoisomers or their salts, or their borane complexes, where: R₄ and R₄′ are equal radicals; or R₄ and R₄′ form, together with the P atom to which they are bound, a 5 to 12 known membered monocyclic, bicyclic, bridged or fused polycyclic ring. In a more preferred embodiment, R₄ and R₄′ are equal radicals.

In a particular embodiment, the bridged or fused “polycyclic” rings are those having 2 or 3 rings. In a more preferred embodiment the bridged polycyclic ring is adamantyl.

In another particular embodiment, the ligands of formula (I) or any of its stereoisomers are those where: R₁ is C₁-C₄ alkyl or substituted or unsubstituted phenyl; R₂ is C₁-C₄ alkyl; R₃ is hydrogen or C₁-C₄ alkyl; and R₄ and R₄′ are C₁-C₄ alkyl.

In a preferred embodiment the ligands of formula (I) or any of its stereoisomers are those where: R₁ is methyl or substituted or unsubstituted phenyl; R₂ is tert-butyl; R₃ is hydrogen or C₁-C₄ alkyl; and R₄ and R₄′ are tert-butyl.

In a more preferred embodiment the ligands of formula (I) or any of its stereoisomers are those selected from the following list:

(P_{R})-(tert-butylmethylphosphino)(di-tert-butylphosphino)amine (Ia; R₁=methyl, R₂=tert-butyl, R₃=hydrogen, R₄=tert-butyl, R₄′=tert-butyl);

(P_{S})-(tert-butylmethylphosphino)(di-tert-butylphosphino)amine (I′a; R₁=methyl, _R₂=tert-butyl, R₃=hydrogen, R₄=tert-butyl, R₄′=tert-butyl);

(P_{S})-(tert-butylphenylphosphino)(di-tert-butylphosphino)amine (Ib; R₁=phenyl, R₂=tert-butyl, R₃=hydrogen, R₄=tert-butyl, R₄′=tert-butyl); (P_{R})— (tert-butylphenylphosphino)(di-tert-butylphosphino)amine (I′ b; R₁=phenyl, R₂=tert-butyl, R₃=hydrogen, R₄=tert-butyl, R₄′=tert-butyl);(P_{R})—N-(tert-butylphenylphosphino)-N-(diphenylphosphino)methylamine (Ic; R₁=tert-butyl, R₂=phenyl, R₃=methyl, R₄=phenyl, R₄′=phenyl);

(P_{S})—N-(tert-butylphenylphosphino)-N-(diphenylphosphino)methylamine (I′ c; R₁=tert-butyl, R₂=phenyl, R₃=methyl, R₄=phenyl, R₄′=phenyl);

(P_{S})—N-(tert-butylmethylphosphino)-N-(diphenylphosphino)methylamine (Id; R₁=tert-butyl, R₂=methyl, R₃=methyl, R₄=phenyl, R₄′=phenyl);

(P_{R})—N-(tert-butylmethylphosphino)-N-(diphenylphosphino)methylamine (I′d; R₁=tert-butyl, R₂=methyl, R₃=methyl, R₄=phenyl, R₄′=phenyl);

(P_{S})—N-(tert-butylmethylphosphino)-N-(di-ortho-tolylphosphino)methylamine (Ie; R₁=tert-butyl, R₂=methyl, R₃=methyl, R₄=2-methylphenyl, R₄′=2-methylphenyl);

(P_{R})—N-(tert-butylmethylphosphino)-N-(di-ortho-tolylphosphino)methylamine (I′e; R₁=tert-butyl, R₂=methyl, R₃=methyl, R₄=2-methylphenyl, R₄′=2-methylphenyl);

(P_{S})—N-(tert-butylmethylphosphino)-N-(dicyclohexylphosphino)methylamine (If; R₁=tert-butyl, R₂=methyl, R₃=methyl, R₄=cyclohexyl, R₄′=cyclohexyl); and

(P_{R})—N-(tert-butylmethylphosphino)-N-(dicyclohexylphosphino)methylamine (I′f; R₁=tert-butyl, R₂=methyl, R₃=methyl, R₄=cyclohexyl, R₄′=cyclohexyl).

As it will be shown in detail in the examples, complete selectivity can be achieved even when very low amounts of catalyst (i.e., 0.3 mol %) are used. These good results are obtained either by using catalysts prepared beforehand, or when the active ligand-metal species is generated in situ by mixing the ligand-metal complex and an organic base.

Also form part of the invention enantiomerically enriched ligands of formula (I) wherein R₁, R₂, R₄ and R₄′ are independently selected from a 5 to 6 membered heterocyclic monocyclic ring unsubstituted or substituted with one or more groups Rₐ, a 6 to 12 membered bridged heterocyclic polycyclic ring unsubstituted or substituted with one or more groups Rₐ, and a 8 to 12 membered fused heterocyclic polycyclic ring unsubstituted or substituted with one or more groups Rₐ, being the ring saturated, partially unsaturated or aromatic, and being the ring attached to the P through a carbon atom; R₃ and Rₐ are as mentioned above; R₁, R₂ are different radicals; and if R₄ and R₄′ are different, R₁ is equal to R₄, and R₂ is equal to R₄′, then the chirality of the phosphorus atoms is not RS or SR.

Enantiomerically enriched ligands of formula (I) or any of its stereoisomers, or their salts, or their borane complex as defined above, can be prepared by a process which comprises: (a) reacting an enantiomerically enriched compound of formula (III) or formula (III′) in the presence of a strong base, with the compound of formula (IV), to yield a aminodiphosphine intermediate borane compound; and (b) reacting the aminodiphosphine intermediate obtained in step (a) with a base or an acid in order to remove the borane group of the aminodiphosphine intermediate borane compound to yield the corresponding enantiomerically enriched compound of formula (I) or any of its stereoisomers, which is isolated in form of free base or as a salt.

Optionally, the ligand of formula (I) or any of its stereoisomers in form of free base can be converted into an acceptable salt thereof in any of its tautomeric forms by treatment with the corresponding acid. The formation of said salts may be carried out by treatment of the ligand of formula (I) or any of its stereoisomers with a sufficient amount of the desired acid. Both organic and inorganic acid salts may be used. Examples of inorganic acids include hydrochloric acid, hydrobromic acid, hydroiodic acid, nitric acid, perchloric acid, sulfuric acid or phosphoric acid. Examples of organic acids include methansulfonic acid, trifluoromethansulfonic acid, ethansulfonic acid, benzenesulfonic acid, p-toluenesulfonic acid, fumaric acid, citric acid, oxalic acid, acetic acid and maleic acid, among others.

Optionally, the ligand of formula (I) or any of its stereoisomers isolated in form of salt can be converted into the free base by reaction with a base. Examples of appropriate bases include alkaline metal carbonates such as sodium carbonate, or a tertiary organic amine such as triethylamine, metal hydrides such as sodium hydride, potassium hydride, or calcium hydride, and alkaline or alkaline earth metal C₁-C₄ alkoxy such as sodium methoxide, sodium ethoxide, or potassium tert-butoxide among others.

In a preferred embodiment the strong base used to prepare the compounds of formula (I) or any of its stereoisomers, or their salts is selected from the group consisting of n-butyl lithium, tert-butyl lithium, methyl lithium, metal hydrides selected from the group consisting of sodium hydride, potassium hydride, or calcium hydride.

Optionally, the ligand of formula (I) or any of its stereoisomers isolated in form of borane complex can be converted into the free base. In a particular embodiment the removal of the borane group of the intermediate aminodiphosphine borane complex is carried out by the addition of an acid such as hydrogen chloride or HBF₄.OMe₂.

In another particular embodiment the removal of the borane group of the intermediate aminodiphosphine borane complex is carried out by the addition of a base such as 1,4-diazabicyclo[2.2.2]octane (DABCO), N,N,N′,N′-tetramethyl-1,2-ethylenediamine (TMEDA), 1,8-diazabicyclo[5.4.0.]undec-7-ene (DBU) or diethylamine, or combinations thereof.

Enantiomerically enriched compounds of formula (III) or formula (III′) where R₃ is hydrogen can be prepared by a process which comprises: (i) reacting an enantiomerically enriched compound of formula (V) or alternatively of formula (V′), with a solution of an alkaline metal selected from Li and Na, and ammonia; or alternatively reacting with hydrogen or a hydrogen source in the presence of a metal catalyst selected from the group consisting of Pd, Pd on carbon and Pd(OH)₂.

Preferably, the enantiomerically enriched compounds of formula (III) or formula (III′) where R₃ is hydrogen can be prepared by a process which comprises: (i) reacting an enantiomerically enriched compound of formula (V) or formula (V′), with a solution of an alkaline metal selected from Li and Na, and ammonia.

The enantiomerically enriched compounds of formula (III) or formula (III′) where R₃ is C₁-C₄ alkyl can be prepared by a process which comprises: (ii) (a) reacting an enantiomerically enriched compound of formula (V) or alternatively of formula (V′) as defined above with R₃X in the presence of a strong base; and (b) reacting the resulting compound of step (a) with a solution of an alkaline metal selected from Li and Na, and ammonia; or alternatively reacting with hydrogen or a hydrogen source in the presence of a metal catalyst selected from the group consisting of Pd, Pd on carbon and Pd(OH)₂.

Preferably, the enantiomerically enriched compounds of formula (III) or formula (III′) where R₃ is C₁-C₄ alkyl can be prepared by a process which comprises: (a) reacting an enantiomerically enriched compound of formula (V) or formula (V′) with R₃X in the presence of a strong base; and (b) reacting the resulting compound of step (a) with a solution of an alkaline metal selected from Li and Na, and ammonia.

In a preferred embodiment ligands of formula (III) or formula (III′) have an ee. equal or greater than 95%. In another preferred embodiment ligands of formula (III) or formula (III″) have an ee. equal or greater than 98%. In a still more preferred embodiment, ligands of formula (III) or formula (III′) have an ee. equal or greater than 99%.

In a preferred embodiment, the alkaline metal is lithium. In another preferred embodiment the strong base is selected from the group consisting of n-butyl lithium, tert-butyl lithium, methyl lithium, and metal hydrides selected from the group consisting of sodium hydride, potassium hydride, and calcium hydride. Preferably, X in the compound of formula R₃X is iodide.

In a particular embodiment, the alkaline metal is lithium, the strong base is n-butyl lithium, and X is iodide.

As it is illustrated in the Scheme 1, the process for preparing the compound of formula (V) or formula (V′) comprises reacting a solution of a chlorophosphines with a chiral amine in the presence of an amine as a base, followed by the addition of the borane complex to the reaction mixture. The obtained mixture was quenched with water and the enantiomerically enriched aminomonophosphine of formula (V) or formula (V′) was isolated.

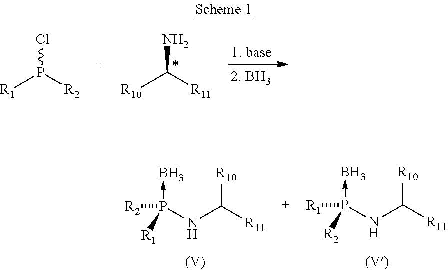

In comparison with the process for obtaining the intermediate monophosphines PCP described in the state of the art, the use of chiral amines allows the formation of diastereomeric mixtures of the compounds of formula (V) or formula (V′). The separation of the above-obtained diastereomer of compounds of formula (V) or (V′) can be made by crystallization methods. Each diastereomer have different physical and chemical properties which afford its easy separation by fractional crystallization. The separation of the said diastereomer may be carried out by treating the diastereomeric mixture of compounds of formula (V) or formula (V′) with a polar solvent. Examples of appropriate solvents are alcohols such as ethanol, methanol or isopropanol.

The starting chlorophosphines and chiral amines are commercially available or can be prepared by any method known in the state of the art.

Enantiomerically enriched compounds of formula (III) and formula (III′) and compounds of formula (V) or (V) are intermediates useful for the preparation of enantiomerically enriched ligands of formula (I) or any of its stereoisomers any of its stereoisomers. Thus, compounds of formula (III) or formula (III′) and compounds of formula (V) or (V′), or their salts are also part of the invention, with the proviso that compounds of formula (V) or (V′) is not a compound of the following list:

Borane complex of (S)—P-tert-butyl-P-phenyl-N—[(S)-1-phenylethyl] phosphinamine (V; R₁=phenyl, R₂=tert-butyl, R₁₀=methyl, R₁₁=phenyl);

Borane complex of (R)—P-iso-butyl-P-tert-butyl-N—[(S)-1-phenylethyl] phosphinamine (V; R₁=iso-butyl, R₂=tert-butyl, R₁₀=methyl, R₁₁=phenyl);

Borane complex of (S)—P-phenyl-P-trimethylphenyl-N—[(S)-1-phenylethyl] phosphinamine (V; R₁=phenyl, R₂=2,4,6-trimethylphenyl, R₁₀=methyl, R₁₁=phenyl); and

Borane complex of (S)—P-tert-butyl-P-phenyl-N-[(1-carbomethoxy-2-methyl)propyl] phosphinamine (V; R₁=phenyl, R₂=tert-butyl, R₁₀=iso-butyl, R₁₁=COOCH₃).

In a preferred embodiment, the enantiomerically enriched compounds of formula (III) or formula (III′) are those where: R₁, and R₂ are different radicals independently selected from the group consisting of substituted or unsubstituted C₁-C₄ alkyl, a substituted or unsubstituted 5 to 6 membered carbocyclic monocyclic ring, and a substituted or unsubstituted 6 to 12 membered bridged carbocyclic polycyclic ring. In a more preferred embodiment, the 5 to 6 membered carbocyclic monocyclic ring is selected from phenyl and cyclohexyl, and the 6 to 12 membered bridged carbocyclic polycyclic ring is adamantyl. In a still more preferred embodiment, the C₁-C₄ alkyl, the cyclohexyl, and the adamantyl are unsubstituted. In a still more preferred embodiment, the C₁-C₄ alkyl is methyl or tert-butyl.

In a particular embodiment, the enantiomerically enriched compounds of formula (III) or formula (III′) are those where: R₁ is C₁-C₄ alkyl; R₂ is C₁-C₄ alkyl or substituted or unsubstituted phenyl and R₃ is hydrogen or C₁-C₄ alkyl.

In a more preferred embodiment the enantiomerically enriched compounds of formula (III) or formula (III′) are those selected from the following list:

Borane complex of (P_{R})—P-tert-butyl(phenyl)phosphinamine (IIIa; R₁=tert-butyl, R₂=phenyl, R₃=hydrogen);

Borane complex of (P_{R})—P-tert-butyl-P-methylphosphinamine (IIIb; R₁=tert-butyl, R₂=methyl, R₃=hydrogen);

Borane complex of (P_{R})—P-tert-butyl-N,P-dimethylphosphinamine (IIIc; R₁=tert-butyl, R₂=methyl, R₃=methyl); and

Borane complex of (P_{R})—P-tert-butyl-N-methyl-P-phenylphosphinamine (IIId; R₁=tert-butyl, R₂=phenyl, R₃=methyl).

In a preferred embodiment, the compounds of formula (IV) are those where: R₄ and R₄′ are radicals independently selected from the group consisting of substituted or unsubstituted C₁-C₄ alkyl, a substituted or unsubstituted 5 to 6 membered carbocyclic monocyclic ring, and a substituted or unsubstituted 6 to 12 membered bridged carbocyclic polycyclic ring. In a more preferred embodiment, the 5 to 6 membered carbocyclic monocyclic ring is selected from phenyl and cyclohexyl, and the 6 to 12 membered bridged carbocyclic polycyclic ring is adamantyl. In a still more preferred embodiment, the C₁-C₄ alkyl, the cyclohexyl, and the adamantyl are unsubstituted. In a still more preferred embodiment, the C₁-C₄ alkyl is methyl or tert-butyl.

In a particular embodiment, the compounds of formula (IV) are those where: R₄ and R₄′ are equal radicals.

In a preferred embodiment, the compounds of formula (IV) are those selected from the following list: di-tert-butylchlorophosphine (IV; R₄=tert-butyl, R₄′=tert-butyl); diphenylchlorophosphine (IV; R₄=phenyl, R₄′=phenyl); di-ortho-tolylchlorophosphine (IV; R₄=2-methylphenyl, R₄′=2-methylphenyl); and dicyclohexylchlorophosphine (IV; R₄=cyclohexyl, R₄′=cyclohexyl).

In another preferred embodiment, the enantiomerically enriched compounds of formula (V) or formula (V′) are those where: R₁, and R₂ are different radicals independently selected from the group consisting of substituted or unsubstituted C₁-C₄ alkyl, a substituted or unsubstituted 5 to 6 membered carbocyclic monocyclic ring, and a substituted or unsubstituted 6 to 12 membered bridged carbocyclic polycyclic ring. In a more preferred embodiment, the 5 to 6 membered carbocyclic monocyclic ring is selected from phenyl and cyclohexyl, and the 6 to 12 membered bridged carbocyclic polycyclic ring is adamantyl. In a still more preferred embodiment, the C₁-C₄ alkyl, the cyclohexyl, and the adamantyl are unsubstituted. In a still more preferred embodiment, the C₁-C₄ alkyl is methyl or tert-butyl.

In a particular embodiment, the enantiomerically enriched compounds of formula (V) or formula (V′) are those where: R₁ is C₁-C₄ alkyl; R₂ is C₁-C₄ alkyl or substituted or unsubstituted phenyl; R₁₀ is a 5 to 6 membered monocyclic ring or a 6 to 12 membered bridged bicyclic ring; R₁₁ is COR₁₂, and R₁₂ is NR₁₃R₁₄.

In a more preferred embodiment, the enantiomerically enriched compounds of formula (V) or formula (V′) are those selected from the following ones:

Borane complex of (P_{R})—P-tert-butyl-P-phenyl-N—[(S)-1-(naphthalen-1-yl)ethyl] phosphinamine (Va; R₁=tert-butyl, R₂=phenyl, R₁₀=naphtyl, R₁₁=methyl); and

Borane complex of (2R)-2-[(P_{S})-tert-butyl(methyl)phosphinoamino]-2-phenyl-acetamide (Vb; R₁=tert-butyl, R₂=methyl, R₁₀=phenyl, R₁₁=CONH₂);

As mentioned above compounds of formulas (III), (III′), (V), or (V′) can be in form of salts. The salts can be prepared by conventional methods, for instance, by treatment of the free compound with the corresponding acid. Both organic and inorganic acid salts may be used. Examples of appropriate acids are the same as described for compounds (I) or any of its stereoisomers.

The compound which comprises an enantiomerically enriched ligand of formula (I) or any of its stereoisomers of the present invention and a metal complex of formula [M^{a+}(L₁)ₘ(L₂)ₙ](A^{b-}), where the metal of the metal complex being bound to the ligand through the phosphorus atoms are also part of the invention. The phosphorus atoms of the ligands of the present invention strongly and diastereoselectivily coordinate with the metal of the above-mentioned metal complex.

In a preferred embodiment, the metal is Ru, Rh or Ir. More preferably, the metal is Rh.

The enantiomerically enriched ligands of formula (I) or any of its stereoisomers maintain their ee. during the complexion with the metal complex. Thus, in a preferred embodiment ligands of formula (I) or any of its stereoisomers bounded to the metal complex have an ee. equal or greater than 95%. In another preferred embodiment, ligands of formula (I) or any of its stereoisomers bounded to the metal complex have an ee. equal or greater than 98%. In a still more preferred embodiment, ligands of formula (I) or any of its stereoisomers bounded to the metal complex have an ee. equal or greater than 99%.

In a preferred embodiment, the metal complex is [Rh(COD)₂] A where A is an anion selected from the group consisting of OTf⁻, PF₆⁻, BE₄⁻, SbF₆, and ClO₄⁻. In a more preferred embodiment, the metal complex is [Rh(COD)₂]BF₄.

The compound which comprises an enantiomerically enriched ligand of formula (I) or any of its stereoisomers of the present invention and a metal complex of formula [M^{a+}(L₁)ₘ(L₂)ₙ](A^{b-}), can be prepared by a process which comprises reacting an enantiomerically enriched ligand of formula (I) or any of its stereoisomers as defined above with a metal complex of formula [M^{a+}(L₁)ₘ(L₂)ₙ](A^{b-}).

When the enantiomerically enriched ligands of formula (I) or any of its stereoisomers are in form of salts, they can be converted into their free bases by treatment with a base. Examples of appropriate bases are sodium carbonate, potassium carbonate, sodium hydrogen carbonate, potassium hydrogen carbonate, sodium sulfate, potassium sulfate, triethylamine, and sodium hydroxide.

The compound which comprises an enantiomerically enriched ligand of formula (I) or any of its stereoisomers of the present invention and a metal complex of formula [M^{a+}(L₁)ₘ(L₂)ₙ](A^{b-}), are useful as catalyst for asymmetric reactions. Thus, the use of the compounds mentioned above as catalyst for asymmetric reactions is also part of the invention.

Preferred asymmetric reactions are those in which the compound obtained have an ee. equal or greater than 95%. Another preferred asymmetric reaction are those in which the compound obtained have an ee. equal or greater than 98%. Still more preferred asymmetric reactions are those in which the amino acids obtained have an ee. equal or greater than 99%.

The chiral catalyst of the present invention can be used in some representative catalytic asymmetric reactions. In a particular embodiment the asymmetric reaction is selected from the group consisting of hydrogenation, hydrosilylation and Michael reactions. In a preferred embodiment the asymmetric reaction is an asymmetric hydrogenation reaction.

As it is shown in the examples, the compounds as mentioned above can be prepared and isolated by the reaction of an enantiomerically enriched ligand of formula (I) or any of its stereoisomers with the metal complex as a solid prior to being used (cf. Example 10); or it can be prepared in situ in the reaction mixture just before their use in the asymmetric hydrogenation reaction (cf. Example 13 Method 2). Their preparation in situ is particularly advantageous since it reduce the number of synthetic steps avoiding their air-exposure during their isolation. It is also advantageous because it allows their use directly from the reaction mixture.

Preferably, the compound mentioned above is used as catalyst in asymmetric hydrogenation reactions of amino acid compounds. In a preferred embodiment, the amino acid compounds thus prepared are alpha-amino acids as it is illustrated in the Scheme 2.

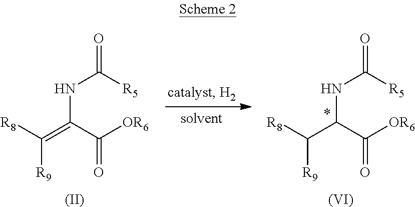

In another preferred embodiment, the amino acid compounds thus prepared are beta-amino acids. These beta-amino acids can be substituted in the alpha or beta position of the carboxylic acid by substituted or unsubstituted C₁-C₄ alkyl. In a preferred embodiment, C₁-C₄ alkyl is selected from the group consisting of methyl and benzyl.

As it is illustrated in the amino acids of formula (VI), (VIII) and (X) thus prepared, a large functional group tolerance can be present in the starting material.

Asymmetric reactions of beta-amino acids from starting materials of formula (VII) and formula (IX) are illustrated in Scheme 3.

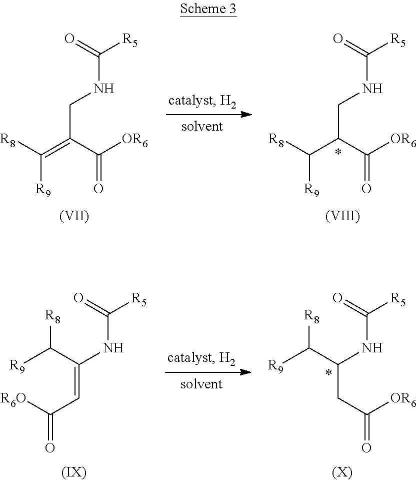

In the previous formulas (II), (VI), (VII), (VIII), (IX) and (X), R₅ is selected from the group consisting of C₁-C₄ alkyl and OR₇; R₆ and R₇ are independently selected from the group consisting of C₁-C₄ alkyl; and R₈ and R₉ are independently selected from the group consisting of C₁-C₄ alkyl, phenyl C₁-C₄ alkyl, C₂-C₄ alkenyl, a 5 to 6 membered monocyclic ring, a 6 to 12 membered bridged polycyclic ring, and 8 to 12 membered fused polycyclic ring, being the ring saturated, partially unsaturated or aromatic, wherein R₈ and R₉ may be optionally substituted with one or more groups independently selected from the group consisting of C₁-C₄ alkyl, halo C₁-C₄alkyl, halogen, C₁-C₄alkoxy, halo C₁-C₄alkoxy, C₁-C₄alkylthio and CN.

In a more preferred embodiment, compounds of formula (II) are those selected from the following list:

(Z)-methyl 2-acetamido-3-phenylacrylate (IIa; R₅=—CH₃, R₆=methyl, R₈=phenyl, R₉=hydrogen);

(Z)-methyl 2-acetamido-3-phenylbut-2-enoate (IIb; R₅═CH₃, R₆=methyl, R₈=phenyl, R₉=methyl);

(Z)-methyl 2-acetamidobut-2-enoate (IIc; R₅═CH₃, R₆=methyl, R₈=methyl, R₉=hydrogen); and

methyl 2-acetamido-3-methylbut-2-enoate (IId; R₅═CH₃, R₆=methyl, R₈=methyl, R₉=methyl).

In another more preferred embodiment, compounds of formula (VII) and compound of formula (IX) are those selected from the following ones:

(E)-methyl 2-(acetamidomethyl)-3-phenylacrylate (VIIa; R₅=—CH₃, R₆=methyl, R₈=phenyl, R₉=hydrogen); and

(E)-methyl 3-acetamidobut-2-enoate (IXa; R₅=CH₃, R₆=methyl, R₈=hydrogen, R₉=hydrogen).

The process for performing the asymmetric hydrogenation reaction defined above comprises reacting a prochiral or chiral olefinic compound in the presence of the compound which comprises an enantiomerically enriched ligand of formula (I) or any of its stereoisomers of the present invention and a metal complex of formula [M^{a+}(L₁)ₘ(L₂)ₙ](A^{b-}), under hydrogen pressure, to produce an optically active compound. The starting olefinic compounds can be prochirals when there are no chiral atoms in their structure, or they can be chiral compounds when there is another chiral atom or chiral atoms in their structure. Additionally, the hydrogen pressure can be achieved by the addition of hydrogen or produced from a hydrogen source. Examples of hydrogen donors include ammonium formate, formic acid or isopropanol. Preferably the hydrogen donor is ammonium formate.

In a preferred embodiment the prochiral or chiral olefin are selected from the group consisting of (II), (VII) or (IX).

In a particular embodiment, the compound which comprises an enantiomerically enriched ligand of formula (I) or any of its stereoisomers of the present invention and a metal complex of formula [M^{a+}(L₁)ₘ(L₂)ₙ](A^{b-}), under an appropriate hydrogenation reaction conditions allows the interchange of the ligands L₁ and L₂ of the metal complex with the solvent used in the reaction. Then, the solvent can be displaced by the olefin of the formula (II), (VII) or (IX). Depending on which enantiomer of the enantiomerically enriched ligand (I) or any of its stereoisomers is used, the asymmetric hydrogenation reaction generates the (R)-enantiomer or (S)-enantiomer of compounds of formula (VI), (VIII) or (X) with a high enantiomeric excess.

Therefore, preferred asymmetric hydrogenation reactions are those in which the amino acids obtained have an ee. equal or greater than 95%. Another preferred asymmetric hydrogenation reactions are those in which the amino acids obtained have an ee. equal or greater than 98%. Still more preferred asymmetric hydrogenation reactions are those in which the amino acids obtained have an ee. equal or greater than 99%.

Typically, an asymmetric hydrogenation reaction depends on the substrate-to-catalyst molar ratio, the hydrogen pressure, reaction temperature and solvent. The substrate-to-catalyst molar ratio usually exceeds about 100:1, but in the asymmetric hydrogenation reactions of the present invention the molar ratio of compounds (II), (VII) or (IX), and the compound which comprises an enantiomerically enriched ligand of formula (I) or any of its stereoisomers and a metal complex of formula [M^{a+}(L₁)ₘ(L₂)ₙ](A^{b-}) is about 50:1. Preferably, the molar ratio is about 30:1; even more preferably about 300:1.

The asymmetric hydrogenation reactions of the present invention are carried out under hydrogen pressure, and at low temperature, preferably at room temperature. In a particular embodiment, the asymmetric hydrogenation reaction is carried out under 3 bar (43 psi) of hydrogen. This selection of reaction conditions is particularly advantageous since it allows a reduction in the reaction time. Thus, the complete conversion of the olefin of formula (II), (VII) or (IX) into the amino acid compound of formula (VI), (VIII) or (X) respectively is carried out in 4 hours.

A great variety of organic solvents and their mixture can be used in the asymmetric hydrogenation reactions of the present invention, including protic, aprotic polar or aromatic solvents. Examples of appropriate protic solvents include alcohols, such as methanol (MeOH), ethanol and isopropanol. Examples of appropriate aprotic polar solvents include tetrahydrofurane (THF), dichloromethane, and acetone. And, examples of appropriate aromatic solvents include toluene, trifluorotoluene and chlorobenzene. In a preferred embodiment, the solvent used in the asymmetric hydrogenation reactions of the present invention is methanol or their mixture. In a more preferred embodiment the solvent used in the asymmetric hydrogenation reaction is a mixture of methanol and THF.

Throughout the description and claims the word “comprises” and variations of the word, are not intended to exclude other technical features, additives, components or steps. Additional objects, advantages and features of the invention will become apparent to those skilled in the art upon examination of the description or may be learned by practice of the invention. The following examples and drawings are provided by way of illustration, and they are not intended to be limiting of the present invention.

### EXAMPLES

The following abbreviations are used in the below examples:

Et₃N: triethylamine

Et₂O: diethyl ether

EtOAc: Ethyl acetate

MeOH: methanol

t-BuOH: tert-butanol

THF: tetrahydrofuran

### Example 1

### Preparation of borane complex of (R)—P-tert-butyl-P-phenyl-N—[(S)-1-(naphthalen-1-yl)ethyl] phosphinamine. (Va; R₁=tert-butyl, R₂=phenyl, R₁₀=naphtyl, R₁₁=methyl)

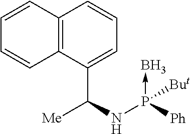

To a solution of (S)-1-(1-naphthyl)ethylamine (0.75 mL, 4.68 mmol) and Et₃N (0.65 mL, 4.68 mmol) in toluene (12 mL) was added tert-butylphenylchlorophosphine (1.0 mL, 4.68 mmol) at room temperature. The mixture was stirred overnight at room temperature. Then borane dimethylsulfide complex (1.0 mL, 9.36 mmol) was added and the reaction was stirred for 20 min. The reaction was quenched with water at 0° C. and extracted with EtOAc. The mixture was purified chromatography (SiO₂, Hexane:EtOAc gradient) to give 1.5 g (91%) of product as a white solid (6:1 mixture of diastereomers). Crystallization in hexane/toluene provided 0.81 g (50%) of the major diastereomer. [α]_{D}+160.3 (c 1.28, CHCl₃); Mp 145-147° C.; IR (KBr): max=3360, 2972, 2388 cm⁻¹; ¹H NMR (400 MHz, CDCl₃): δ 0.50-0.98 (br, 3H, BH3), 1.11 (d, J=14 Hz, 9H), 1.74 (d, J=7 Hz, 3H), 5.34 (m, 1H), 7.11-7.22 (m, 3H), 7.27 (m, 1H), 7.34-7.41 (m, 3H), 7.44 (d, J=8 Hz, 1H), 7.56 (d, J=7 Hz, 1H), 7.73 (d, J=8 Hz, 1H), 7.79 (d, J=8 Hz, 1H), 7.90 (d, J=7 Hz, 1H) ppm; HRMS (ESI): calcd for C₂₂H₂₆NP+H, 336.18757 found 336.18751. Anal Calcd for C₂₂H₂₉BNP: C, 75.66; H, 8.37; N, 4.01. Found C, 75.51; H, 8.52; N, 4.02.

### Example 2

### Preparation of borane complex of (R)-tert-butyl(phenyl)phosphinamine. (IIIa; R₁=tert-butyl, R₂=phenyl, R₃=hydrogen)

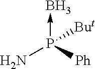

In a two-neck flask was condensed NH₃ (5 mL) at −78° C., then was added Li (8 mg, 1.14 mmol). The mixture was stirred 10 min. and the solution turned blue. Then a solution of borane complex of (R)—P-tert-butyl-P-phenyl-N—[(S)-1-(naphthalen-1-yl)ethyl] phosphinamine (100 mg, 0.28 mmol) in THF (1.0 mL) and tBuOH (0.05 mL) was added dropwise. The solution turned red-orange after 5-10 min. NH₄Cl was added and NH₃ was evaporated at room temperature. At this point H₂O (5 mL) was added, the aqueous layer was washed with 2×5 mL EtOAc, and the combined organic layer was dried with anhydrous magnesium sulfate, filtered, and concentrated under vacuum. The resulting crude was purified by chromatography (SiO₂, Hexane-EtOAc gradient) to give 36 mg (67%) of product as a white solid (99% ee). Mp 103-105° C.; IR (KBr): max=3446, 3342, 2926, 2868, 2375, 2341 cm⁻¹; ¹H NMR (400 MHz, CDCl₃): δ 0.30-1.00 (br, 3H, BH3), 1.10 (d, J=14 Hz, 9H), 1.62 (br, 1H, NH), 2.04 (br, 1H, NH), 7.47 (m, 3H), 7.70 (m, 2H) ppm; MS (ESI, H₂O:CH₃CN (1:1) 1% formic acid) m/z: 182 [(C₁₀H₁₇NP)⁺, 30%], 196 [(M+H)⁺, 65%]; HRMS (ESI): calcd for C₁₀H₁₉BNP+H, 196.1426 found 196.1422.

### Example 3

### Preparation of borane complex of (R)—P-tert-butyl-P-phenyl-N-methyl-N—[(S)-1-(naphthalen-1-yl)ethyl] phosphinamine

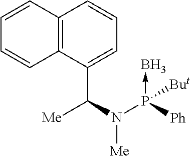

To a solution of borane complex of (R)—P-tert-butyl-P-phenyl-N—[(S)-1-(naphthalen-1-yl)ethyl] phosphinamine (1.5 g, 4.29 mmol) in THF (15 mL) at −78° C. was added butyllithium (1.89 mL of a 2.5 M solution in hexane, 4.72 mmol). The mixture was stirred at this temperature for 15 minutes and then methyl iodide (1.06 mL, 17.18 mmol) was added. The reaction was stirred overnight at room temperature, quenched with water and extracted with EtOAc, the combined organic layer was dried with anhydrous magnesium sulfate, filtered, and concentrated in vacuum. The resulting crude was purified by chromatography (SiO₂, Hexane:EtOAc gradient) to give 1.25 g (81%) of product as a white solid. [α]_{D}+112.2 (c 1.25, CHCl₃); Mp 173-175° C.; IR (KBr): max=3048, 2964, 2390 cm⁻1; ¹H NMR (400 MHz, CDCl₃): δ 0.50-1.10 (br, 3H, BH3), 1.32 (d, J=14 Hz, 9H), 1.75 (d, J=7 Hz, 3H), 2.49 (d, J=6 Hz, 3H), 5.96 (m, 1H), 6.98 (m. 1H). 7.20 (m, 2H), 7.34 (m, 2H), 7.44 (m, 3H), 7.53 (d, J=7 Hz, 1H), 7.79 (d, J=8 Hz, 2H), 8.13 (d, J=9 Hz, 1H) ppm; MS (ESI, H₂O:CH₃CN (1:1) 1% formic acid) m/z: 362 [(M−H)⁺, 6%], 386 [(M+Na)⁺, 65%]; HRMS (ESI): calcd for C₂₃H₃₁BNP+Na: 386.21794 found 386.21774. Anal Calcd for C₂₃H₃₁BNP: C, 76.04; H, 8.60; N, 3.86. Found C, 75.73; H, 8.63; N, 3.61.

### Example 4

### Preparation of borane complex of (R)—P-tert-butyl-N-methyl-P-phenylphosphinamine (IIId; R₁=tert-butyl, R₂=phenyl, R₃=methyl)

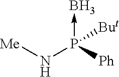

In a two-neck flask was condensed NH₃ (100 mL) at −78° C., then was added Li (91 mg, 13.18 mmol). The mixture was stirred 10 minutes and the solution turned blue. Then a solution of borane complex of (R)—P-tert-butyl-P-phenyl-N-methyl-N—[(S)-1-(naphthalen-1-yl)ethyl] phosphinamine (1.2 g, 3.30 mmol) in THF (12 mL) and tBuOH (0.65 mL) was added dropwise. The solution turned red-orange after 5-10 min. NH₄Cl was added and NH₃ was evaporated at room temperature. At this point H₂O (15 mL) was added, the aqueous layer was washed with 3×15 mL EtOAc, and the combined organic layer was dried with anhydrous magnesium sulfate, filtered, and concentrated in vacuum. The resulting crude was purified by chromatography (SiO₂, Hexane:EtOAc gradient) to give 479 mg (70%) of product as a white solid (99% ee as determined by HPLC). [α]_{D}+87.8 (c 1.08, CHCl₃); MP 124-126° C.; IR (KBr): max=3358, 2926, 2381 cm−1; ¹H NMR (400 MHz, CDCl₃): δ 0.28-1.01 (br, 3H, BH₃), 1.10 (d, J=14 Hz, 9H), 2.71 (d, J=11 Hz, 3H), 7.45 (m, 3H), 7.65 (m, 2H) ppm; HRMS (ESI): calcd for C₁₁H₂₁BNP+H, 210.15775 found 210.15770. Anal Calcd for C₁₁H₂₁BNP: C, 63.19; H, 10.12; N, 6.70. Found C, 63.59; H, 10.28; N, 6.61.

### Example 5

### Preparation of borane complex of (R)—P-tert-butyl-N,P-dimethyl-N—[(S)-1-(naphthalen-1-yl)ethyl] phosphinamine

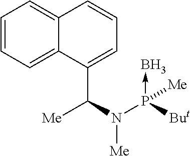

To a solution of methyl-tert-butyl-chlorophosphine (5.71 mmol) in Et₂O (10 mL) at 0° C. was added (R)-1-(1-naphthyl)ethylamine (0.8 mL, 5.14 mmol) and Et₃N (0.8 mL, 5.71 mmol). The mixture was stirred overnight. Then borane dimethylsulfide complex (1.6 mL, 17.31 mmol) was added and the reaction stirred for 1 hour. Then the reaction was quenched with water at 0° C., washed with brine and extracted with EtOAc. The mixture was purified by silica gel chromatography (95:5, hexane:EtOAc) to give 1.2 g of the corresponding phosphinamine borane complex containing triethylamine borane complex. This mixture was solved in THF (35 mL) and at −78° C. was added butyllithium (3.1 mL of a 2.5 M solution in hexane, 7.71 mmol). The mixture was stirred at this temperature for 15 minutes and then methyl iodide (1.3 mL, 20.56 mmol) was added. The reaction was stirred overnight at room temperature, quenched, washed with brine and extracted with EtOAc. The mixture was purified by silica gel chromatography (95:5, hexane:EtOAc) to give 850 mg (52%) of the title compound as a 2.5:1 mixture of diastereomers. The diastereomers could be separated by recrystallization toluene/hexane (96° A) ee as determined by HPLC). Mp: 130-131° C.; [α]_{D}=+130.4 (c=0.5, CHCl₃); IR (film): νₘₐₓ=2284, 2372, 2979, 3047 cm−1; ¹H NMR (400 MHz, CDCl₃): δ 1.31 (d, J=14 Hz, 9H), 1.36 (d, J=8 Hz, 3H), 1.63 (d, J=7 Hz, 3H), 2.53 (d, J=7 Hz, 3H), 5.72 (q, J=7 Hz, 1H), 7.42-7.55 (m, 4H), 7.77 (d, J=8 Hz, 1H), 7.84 (d, J=8 Hz, 1H), 8.37 (d, J=8 Hz, 1H) ppm; HRMS-ESI: m/z calcd for C₁₈H₃₀NBP: 302.2203, found 302.2202 [M+H]⁺. Anal. cald. For C₁₈H₂₉BNP: C, 71.77; H, 9.70; N, 4.65. Found C, 71.66; H, 9.85; N, 4.61.

### Example 6

### Preparation of borane complex of (2R)-2-[(S)-tert-butyl(methyl)phosphinoamino]-2-phenyl-acetamide. (Vb; R₁=tert-butyl, R₂=methyl, R₁₀=phenyl, R₁₁=CONH₂)

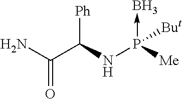

To a solution of methyl-tert-butyl-chlorophosphine (4.2 g, 30.52 mmol) in THF (60 mL) at 0° C. was added Et₃N (4.25 mL, 30.52 mmol) and the mixture was stirred during 10 min. Then (R)-2-phenylglycinamide (4.6 g, 30.52 mmol) was added by portions during 3 h. The mixture was stirred overnight at room temperature. The crude was filtered under N₂ and borane dimethylsulfide complex (3.8 mL, 39.67 mmol) was added to the filtrated solution at 0° C. and the reaction stirred for 30 min. Then the reaction was quenched with water at 0° C., washed with brine and extracted with EtOAc. The combined organic layer was dried with anhydrous magnesium sulfate, filtered, and concentrated in vacuum to give 7.9 g (98%) of desired product as a 1.5:1 mixture of diastereomers. This mixture was crystallized from hot EtOH (40 mL) to afford 3.2 g of major diastereomer (40% yield, 98% de). This fraction was digested with EtOH (20 mL) at reflux to obtain 2.6 g of major diastereomer (33%, 99.9% de). Mp: 194-195° C.; [α]_{D}=−120.6 (c=0.5, CHCl₃); IR (film): νₘₐₓ=1388, 1667, 2369 cm⁻¹; ¹H NMR (400 MHz, CDCl₃): δ 1.14 (d, J=12 Hz, 3H), 1.17 (d, J=12 Hz, 9H), 4.99 (d, J=9 Hz, 1H), 5.47 (br s, 1H), 5.58 (br s, 1H), 7.31-7.40 (m, 5H) ppm; HRMS-ESI: m/z calcd for C₁₃H₂₅ON₂BP: 267.1792, found 267.1791 [M+H]⁺; Anal Calcd for C₁₃H₂₄BN₂OP: C, 58.67; H, 9.09; N, 10.53. Found C, 58.64; H, 9.11; N, 10.17.

### Example 7

### Preparation of borane complex of (R)—P-tert-butyl-N,P-dimethylphosphinamine. (IIIc; R₁=tert-butyl, R₂=methyl, R₃=methyl)

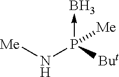

In a two-neck flask was condensed NH₃ (15 mL) at −78° C., then was added Li (13 mg, 2.02 mmol). The mixture was stirred 10 minutes and the solution turned blue. Then was added a solution of borane complex of (R)—P-tert-butyl-N,P-dimethyl-N—[(S)-1-(naphthalen-1-yl)ethyl] phosphinamine (138 mg, 0.50 mmol) in THF (2 mL) and tBuOH (0.10 mL) dropwise. The solution turned red-orange after 5-10 min. NH₄Cl was added and NH₃ was evaporated at room temperature. At this point H₂O (5 mL) was added, the aqueous layer was washed with 3×5 mL EtOAc, and the combined organic layer was dried with anhydrous magnesium sulfate, filtered, and concentrated in vacuum. The resulting crude was purified by silica gel in a combiflash chromatography (Hexane:EtOAc gradient) to give 59 mg (80%) of product as a white solid. [α]_{D}-18.9 (c 0.96, CHCl₃); Mp 62-65° C.; IR (KBr): max=3360, 2960, 2374 cm⁻¹; ¹H NMR (400 MHz, CDCl₃): δ 0.43 (qd, J=95 and 15 Hz 3H, BH₃), 1.13 (d, J=14 Hz, 9H), 1.32 (d, J=9 Hz, 3H), 1.51 (br, 1H, NH), 2.68 (d, J=10 Hz, 3H) ppm; HRMS (ESI): calcd for C₆H₁₆NOP+H, 150.1048 found 150.1048. Anal Calcd for C₁₁H₂₁BNP: C, 49.02; H, 13.03; N, 9.53. Found C, 49.51; H, 13.40; N, 9.48.

### Example 8

### Preparation of borane complex of (S)—P-tert-butyl-P-methylphosphinamine. (III; R₁=tert-butyl; R₂=methyl, R₃=hydrogen

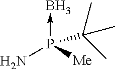

In a two-neck flask was condensed NH₃ (65 mL) at −78° C., then was added Li (64 mg, 9.85 mmol). The mixture was stirred 10 minutes and the solution turned blue. Then was added a solution of (2R)-2-(tert-butyl(methyl)phosphinoamino)-2-phenyl-acetamide borane complex (655 mg, 2.46 mmol) in THF (8 mL) and tBuOH (0.47 mL) dropwise. After 10 minutes NH₄Cl was added and NH₃ was evaporated at room temperature. At this point H₂O (5 mL) was added, the aqueous layer was washed with 3×5 mL EtOAc, and the combined organic layer was dried with anhydrous magnesium sulfate, filtered, and concentrated in vacuum. The resulting crude was purified by silica gel in a combiflash chromatography (Hexane:EtOAc gradient) to give 236 mg (72%) of product as a white solid. [α]_{D}: +18 (c 0.41, CHCl₃); Mp 162-164° C.; IR (KBr): max=3433, 3341, 2967, 2867, 2374 cm⁻¹; ¹H NMR (400 MHz, CDCl₃): δ0.47 (qd, J=80 and 16 Hz 3H, BH₃), 1.14 (d, J=14 Hz, 9H), 1.35 (d, J=9 Hz, 3H), 1.74 (br, 1H, NH) ppm; MS (ESI, H₂O:CH₃CN (1:1) 1% formic acid) m/z: 120 [(M+H−BH₃)⁺, 2%], 132 [(M−H)⁺, 3%], 263 [(C₁₀H₃₀B₂N₂P₂+H)⁺, 100%]; HRMS (ESI): calcd for C₅H₁₇BNP−H, 132.1113 found 132.1108.

### Example 9

### Preparation of aminodiphosphine hydrochloride salt (I′a R₁=methyl, R₂=tert-butyl, R₄=tert-butyl, R₄′=tert-butyl)

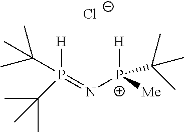

To a solution of borane complex of (S)—P-tert-butyl-P-methylphosphinamine (125 mg, 0.89 mmol) in THF (2 mL) at 0° C. was added butyllithium (0.4 mL of a 2.5 M solution in hexane, 0.98 mmol). The mixture was stirred 20 minutes. Then di-tert-butylchlorophosphine (0.2 mL, 1.02 mmol) was added and the mixture was stirred at 65° C. for 8 hours. The crude was purified by chromatography (SiO₂, Hexane:EtOAc gradient) to give 118 mg (48%) of the intermediate diphosphinoamine borane complex as a white solid. To a solution of diphosphinoamine borane complex (118 mg, 0.42 mmol) in MeOH (8 mL) was added hydrogen chloride (1.68 mL of a 1.25 M solution in methanol, 2.1 mmol) at room temperature. The mixture was stirred overnight at 65° C., and then the solvent was removed in vacuo to provide 133 mg (95%) of the desired chloride salt. IR (KBr): max=3390, 2952, 2870, 1475, 1273 cm⁻¹; ¹H NMR (300 MHz, CDCl₃): δ 1.23 (d, J=18 Hz, 9H), 1.31 (d, J=19 Hz, 9H), 1.37 (d, J=19 Hz, 9H), 1.95 (d, J=13 Hz, 3H), 2.57 (br, 1H, NH), 6.59 (d, J=61, 1H), 8.18 (d, J=45, 1H) ppm.

### Example 10

### Preparation of Aminodiphosphine Rhodium complex

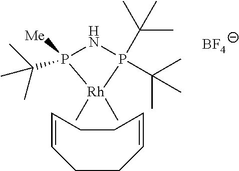

A solution of aminodiphosphine hydrochloride salt of Example 9 (229 mg, 0.68 mmol) in EtOAc (5 mL) was washed with degassed Na₂CO₃ aqueous solution (5 mL). The organic layer was separated and the aqueous layer was extracted with EtOAc (2×5 ml). The solvent was removed in vacuum and the crude was dissolved with CH₂Cl₂ (10 mL). The resulting solution was slowly added to a solution of [Rh (COD)₂]BF₄ in CH₂Cl₂ (2 mL) at room temperature. After addition was complete, the reaction mixture was stirred for 20 min and solvent was removed in vacuum to provide an orange solid. The solid was digested with Et₂O (3×3 mL) to provide 246 mg (65%) of the title compound as an orange solid. IR (film): νₘₐₓ=3277, 2946, 1475, 1056 cm⁻¹; ¹H NMR (400 MHz, CDCl₃): δ1.21 (d, J=16 Hz, 9H), 1.38 (d, J=15 Hz, 9H), 1.40 (d, J=1a Hz, 9H), 1.77 (dd, J=8 and 1Hz, 3H), 2.10-2.30 (m, 4H), 2.36-2.57 (m, 4H), 5.11 (br, 2H), 5.39 (br, 1H), 5.54 (m, 2H) ppm; HRMS-ESI: m/z calcd for C₂₁H₄₃NP₂Rh: 474.19203, found 474.19196.

### Example 11

### Preparation of aminodiphosphine tetrafluoroborate salt. (I′a; R₁=methyl, R₂=tert-butyl, R₄=tert-butyl, R₄′=tert-butyl)

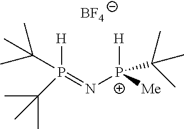

To a solution of the intermediate diphosphinoamine borane complex of Example 9 (45 mg, 0.162 mmol) in MeOH (5 mL) was added HBF₄.EtO₂ (0.11 mL, 0.82 mmol) at room temperature. The mixture was stirred overnight at 65° C., and then the solvent was removed in vacuum to provide 55 mg (77%) of the desired tetrafluoroborate salt. [α]_{D}+39.37 (c 0.48, CHCl₃); IR (KBr): max=2964, 2869, 1475, 1305, 1053 cm⁻¹; ¹H NMR (300 MHz, CDCl₃): δ 1.24 (d, J=18 Hz, 9H), 1.30 (d, J=17 Hz, 9H), 1.33 (d, J=17 Hz, 9H), 1.77 (dd, J=13 and 4 Hz, 3H), 6.57 (dd, J=458 and 6 Hz, 1H), 6.83 (dd, J=477 and 4 Hz, 1H) ppm.

### Example 12

### Preparation of the diborane complex of (S,S)—N,N′-bis(tert-butylmethylphosphino)methylamine (I; R₁=methyl, R₂=tert-butyl, R₃=methyl, R₄=tert-butyl, R₄′=methyl)

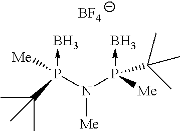

An oven-dried, one-neck, round-bottomed flask equipped with a magnetic stirring bar was charged with the borane complex of (R)—P-tert-butyl-N,P-dimethylphosphinamine (1.09 g, 7.41 mmol). Anhydrous THF (10 mL) was added, and the solution was cooled to 0° C. Then 2.5 M n-BuLi (3.25 mL, 8.14 mmol) was added drop-wise to this solution. After stirring for 10 min at 0° C., a suspension of NaH (194 mg, 8.14 mmol) in THF (10 mL) was added. At this point, a solution of tert-BuMePCl (1.24 g, 8.16 mmol) in THF (5 mL) was added drop-wise at room temperature and the solution was stirred for 30 min. Next, BH₃—SMe₂ (0.91 mL, 9.62 mmol) was added and the mixture was stirred for 20 min. At this point, H₂O (15 mL) was added. The aqueous layer was then washed with 3×15 mL EtOAc, and the combined organic layer was dried with anhydrous magnesium sulfate, filtered, and concentrated under vacuum. The resulting crude product was purified by chromatography (SiO₂, Hexane:EtOAc gradient) to give 777 mg (41%) the undesired meso diphosphine and 610 mg (32%) of the desired C₂ diphosphine as a white solid. [α]_{D}: −65.69 (c 0.65, CHCl₃); Mp 70-73° C.; IR (KBr): max=2952, 2369, 911 cm⁻¹; ¹H NMR (400 MHz, CDCl₃): δ 0.26-1.14 (q, J=92 Hz, 6H, BH₃), 1.25 (m, 18H), 1.67 (m, 6H), 3.11 (t, J=10 Hz, 3H) ppm. ¹³C NMR (100 MHz, CDCl₃): δ 11.8 (d, J_{P}=39 Hz, 2CH₃), 26.6 (d, J_{P}=2 Hz, 6CH₃), 35.0 (d, J_{P}=28 Hz, 2C), 40.3 (t, J_{P}=5 Hz, CH₃) ppm; ³¹P NMR (121 MHz, CDCl₃) δ 98.7 (m, P—BH₃) ppm.

### Example 13

### Preparation of (2S)-Methyl-2-acetamido-3-phenylpropanoate

Method 1: Hydrogenation with Isolated Aminodiphosphine Rhodium Complex

Aminodiphosphine Rhodium complex of example 10 (2.8 mg, 0.005 mmol), (Z)-methyl-2-acetamido-3-phenylpropenoate (36 mg, 0.16 mmol) and MeOH (1.5 mL) were placed in a pressure vessel. The vessel was purged with hydrogen and charged to 3 bar of H₂. The reaction was stirred at room temperature for 4 hours. The crude was filtered on silica gel to remove the catalyst and the solvent was removed under vacuum to yield 35 mg (99%) of the reduction product as a white solid in 99% ee as determined by HPLC. HPLC analysis: CHIRALPACK OJ, 10% IPA-90% heptane, 1.0 mL/min, A=220 nm. t_{(R)} isomer=14.6 min. t_{(S)} isomer=21.8 min.

¹H NMR (400 MHz, CDCl₃): δ 1.98 (s, 3H), 3.13 (m, 2H), 3.73 (s, 3H), 4.88 (m, 1H), 5.90 (br, 1H, NH), 7.09 (m, 2H), 7.28 (m, 3H) ppm.

### Method 2: Hydrogenation Using Aminodiphosphine Rhodium Complex Generated In Situ.

### Step 1: Preparation of Aminodiphosphine Rhodium Complex Solution

To a solution of aminodiphosphine tetrafluoroborate salt of example 11 (25 mg, 0.057 mmol) in MeOH (5 mL) under nitrogen were added [Rh(COD)₂]BF₄ (20 mg, 0.049 mmol) and Et₃N (0.013 mL, 0.098 mmol). The resulting solution was let to stir at room temperature for 1 h. Aliquots (1 mL) of this solution (9.8×10⁻³ M) were taken to employ as catalyst.

### Step 2: Hydrogenation Step

A pressure vessel was charged with methyl-2-acetamido-3-phenylpropenoate (89 mg, 0.41 mmol). The aminodiphosphine catalyst solution in MeOH (1 mL) prepared as described in step 1 was added to the vessel. The reactor was charged to 3 bar of hydrogen and the reaction mixture was stirred at room temperature overnight. The crude was filtrated on silica gel to provide (2S)-methyl-2-acetamido-3-phenylpropanoate 91 mg (99%) as a white solid (99% ee determined by chiral HPLC).

### Example 14

### Preparation of (S)-methyl 3-acetamidobutanoate

A pressure vessel was charged with (E)-methyl 3-acetamidobut-2-enoate (51 mg, 0.33 mmol). A catalyst solution in MeOH (1 mL) prepared as described in example 13 step 1 was added to the vessel. The reactor was charged to 3 bar of hydrogen and the reaction mixture was stirred at room temperature overnight. The crude was filtrated on silica gel to provide (S)-methyl 3-acetamidobutanoate 51 mg (99%) as a white solid (99% ee determined by chiral gas chromatography).

### Example 15

### Preparation of (S)-methyl 3-acetamido-2-benzylpropanoate

A pressure vessel was charged with (E)-methyl 2-(acetamidomethyl)-3-phenylacrylate (77 mg, 0.33 mmol). A catalyst solution in MeOH (1 mL) prepared as described in example 13 step 1 was added to the vessel. The reactor was charged to 3 bar of hydrogen and the reaction mixture was stirred at room temperature overnight. The crude was filtrated on silica gel to provide (S)-methyl 3-acetamido-2-benzylpropanoate 77 mg (99%) as a white solid (88% ee determined by chiral gas chromatography).

Following the same procedure as described in example 13 method 1 or 2, the asymetric hydrogenations on the substrates indicated in Table 1 were carried out:

TABLE 1

| | | Amount | Amount | H₂ | React. | ee | |
|---|---|---|---|---|---|---|---|
| Ex. | Substrate | of substrate | of catalyst | (bar) | time | (%) | Yield |
| 16 | 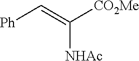 | 18.0 mg, 0.082 mmol | 0.14 mg, 0.25 μmol, 0.30 mol % | 3 | 9 h | 99 | 99 |
| 17 | | 89 mg, 0.41 mmol | 4.10 μmol, 1.0 mol % (prepared in situ) | 3 | overnight | 99 | 99 |
| 18 | 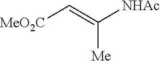 | 19.1 mg, 0.122 mmol | 0.21 mg, 0.37 μmol, 0.30 mol % | 3 | 9 h | 99 | 99 |
| 19 | 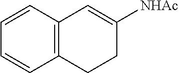 | 62 mg, 0.33 mmol | 9.90 μmol, 3.0 mol % (prepared in situ) | 10 | 4 days | 99 | 99 |

The analytical data for the compounds obtained in the previous examples are:

### Examples 16 and 17

HPLC analysis: Chiralcel OJ-H (25×0.46 cm, Chiral Technologies®), 10% IPA-90% heptane, 1.0 mL/min, λ=220 nm, t_{R}=12.6 min, t_{S}=17.7 min.

### Example 18

GC analysis: Beta-Dex 120 (30 m×0.25 mm×0.25 μm, Supelco®), gradient: 80° C. (3 min)—5° C./min—210° C., 15 psi He, t_{S}=20.5 min, t_{R}=20.7 min

### Example 19

HPLC analysis: CHIRALPACK IA, 5% IPA-95% heptane, 1 mL/min, λ=254 nm. t_{S} isomer=19.2 min, t_{R} isomer=24.6 min.

1. An enantiomerically enriched ligand of formula (I), 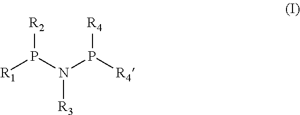
or any of its stereoisomers which are: 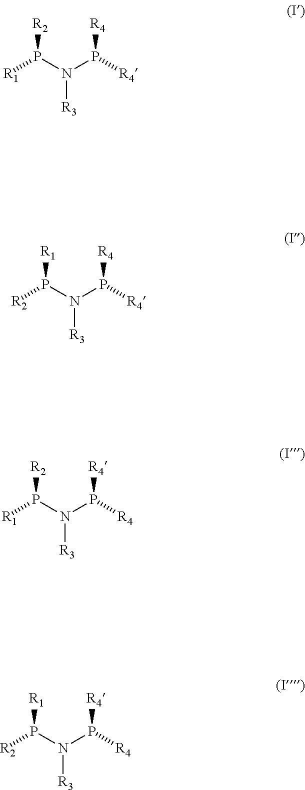
or a salt in any of its tautomeric forms, or borane complex thereof in any of its tautomeric forms, wherein:
R₁, R₂, R₄ and R₄′ are radicals independently selected from the group consisting of C₁-C₄ alkyl unsubstituted or substituted with one or more groups Rₐ, phenyl C₁-C₄ alkyl unsubstituted or substituted with one or more groups Rₐ, C₂-C₄ alkenyl unsubstituted or substituted with one or more groups Rₐ, a 5 to 6 membered carbocyclic monocyclic ring unsubstituted or substituted with one or more groups Rₐ, a 6 to 12 membered bridged carbocyclic polycyclic ring unsubstituted or substituted with one or more groups Rₐ, and a 8 to 12 membered fused carbocyclic polycyclic ring unsubstituted or substituted with one or more groups Rₐ, being the ring saturated, partially unsaturated or aromatic; or alternatively
R₄ and R₄′ form, together with the P atom to which they are bound, a 5 to 12 known membered monocyclic, bicyclic, bridged or fused polycyclic ring, being the ring saturated, partially unsaturated or aromatic; the members of the ring being independently selected from C, N, O, S, and P;
each Rₐ is independently selected from the group consisting of C₁-C₄ alkyl, halo C₁-C₄ alkyl, halogen, C₁-C₄ alkoxy, halo C₁-C₄ alkoxy, C₁-C₄ alkylthio, and CN;
R₃ is hydrogen or C₁-C₄ alkyl;
wherein R₁, R₂ are different radicals; and
if R₄ and R₄′ are different, R₁ is equal to R₄, and R₂ is equal to R₄′, then the chirality of the phosphorus atoms is not RS or SR.

2. The enantiomerically enriched ligand according to claim 1, wherein:
R₁, R₂, R₄ and R₄′ are radicals independently selected from the group consisting of substituted or unsubstituted C₁-C₄ alkyl, a substituted or unsubstituted 5 to 6 membered carbocyclic monocyclic ring, and a substituted or unsubstituted 6 to 12 membered bridged carbocyclic polycyclic ring.

3. The enantiomerically enriched ligand according to claim 2, wherein the 5 to 6 membered carbocyclic monocyclic ring is selected from phenyl and cyclohexyl, and the 6 to 12 membered bridged carbocyclic polycyclic ring is adamantyl.

4. The enantiomerically enriched ligand according to claim 3, wherein the C₁-C₄ alkyl, the cyclohexyl, and the adamantyl are unsubstituted.

5. The enantiomerically enriched ligand according to claim 4, wherein the C₁-C₄ alkyl is methyl or tert-butyl.

6. The enantiomerically enriched ligand according to claim 1, wherein: R₄ and R₄′ are equal radicals.

7. The enantiomerically enriched ligand according to claim 1, wherein: R₁ is C₁-C₄ alkyl or substituted or unsubstituted phenyl; R₂ is C₁-C₄ alkyl; and R₄ and R₄′ are C₁-C₄ alkyl.

8. The enantiomerically enriched ligand according to claim 7, wherein:
R₁ is methyl or substituted or unsubstituted phenyl;
R₂ is tert-butyl;
R₄ and R₄′ are tert-butyl.

9. The enantiomerically enriched ligand according to claim 1, which is selected from the group consisting of:
(P_{R})-(tert-butylmethylphosphino) (di-tert-butylphosphino) amine (Ia; R₁=methyl, R₂=tert-butyl, R₃=hydrogen, R₄ and R₄′=tert-butyl);
(P_{S})-(tert-butylmethylphosphino) (di-tert-butylphosphino) amine (I′a; R₁=methyl, R₂=tert-butyl, R₃=hydrogen, R₄ and R₄′=tert-butyl);
(P_{S})-(tert-butylphenylphosphino) (di-tert-butylphosphino) amine (Ib; R₁=phenyl, R₂=tert-butyl, R₃=hydrogen, R₄ and R₄′=tert-butyl);
(P_{R})-(tert-butylphenylphosphino) (di-tert-butylphosphino) amine (I′b; R₁=phenyl, R₂=tert-butyl, R₃=hydrogen, R₄ and R₄′=tert-butyl);
(P_{R})—N-(tert-butylphenylphosphino)-N-(diphenylphosphino)methylamine (Ic; R₁=tert-butyl, R₂=phenyl, R₃=methyl, R₄ and R₄′=phenyl);
(P_{S})—N-(tert-butylphenylphosphino)-N-(diphenylphosphino)methylamine (I′c; R₁=tert-butyl, R₂=phenyl, R₃=methyl, R₄ and R₄′=phenyl);
(P_{S})—N-(tert-butylmethylphosphino)-N-(diphenylphosphino)methylamine (Id; R₁=tert-butyl, R₂=methyl, R₃=methyl, R₄ and R₄′=phenyl);
(P_{R})—N-(tert-butylmethylphosphino)-N-(diphenylphosphino)methylamine (I′d; R₁=tert-butyl, R₂=methyl, R₃=methyl, R₄ and R₄′=phenyl);
(P_{S})—N-(tert-butylmethylphosphino)-N-(di-ortho-tolylphosphino)methylamine (Ie; R₁=tert-butyl, R₂=methyl, R₃=methyl, R₄ and R₄′=2-methylphenyl);
(P_{R})—N-(tert-butylmethylphosphino)-N-(di-ortho-tolylphosphino)methylamine (I′e; R₁=tert-butyl, R₂=methyl, R₃=methyl, R₄ and R₄′=2-methylphenyl);
(P_{S})—N-(tert-butylmethylphosphino)-N-(dicyclohexylphosphino)methylamine (If; R₁=tert-butyl, R₂=methyl, R₃=methyl, R₄ and R₄′=cyclohexyl); and
(P_{R})—N-(tert-butylmethylphosphino)-N-(dicyclohexylphosphino)methylamine (I′ f; R₁=tert-butyl, R₂=methyl, R₃=methyl, R₄ and R₄′=cyclohexyl).

10. A compound which comprises an enantiomerically enriched ligand of formula (I) or any of its stereoisomers according to claim 1, and a metal complex of formula [M^{a+}(L₁)ₘ(L₂)ₙ](A^{b-}), the metal of the metal complex being bound to the ligand through the phosphorus atoms, wherein:
M is a metal selected from the group consisting of Ru, Rh, Ir, and Cu;
L₁ is a diene selected from the group consisting of 1,5-cyclooctediene, norbornadiene, and 2,5-dimethyl-hexa-1,5-diene;
L₂ is an anionic ligand selected from the group consisting of Cl⁻, Br⁻, I⁻, ⁻CN, OR₁₆, and ⁻R₁₆ or a neutral σ-donor ligand selected from the group consisting of NR₁₆R₁₇R₁₈, R₁₆OR₁₇, R₁₆SR₁₇, CO, and NCR₁₆;
R₁₆, R₁₇ and R₁₈ are independently selected from the group consisting of hydrogen and C₁-C₆ alkyl;
A is an anion selected from the group consisting of OTf⁻, PF₆⁻, BF₄, SbF₆⁻, and ClO₄⁻;
m is an integer from 0 to 3, inclusive;
n is an integer from 0 to 3, inclusive;
m+n is an integer from 0 to 3 inclusive;
b is the number of negative charges of the anion; and
a is the number of positive charges of the metal ion.

11. The compound according to claim 10, wherein the metal complex is [Rh (COD)_{2]BF}₄.

12. (canceled)

13. (canceled)

14. (canceled)

15. (canceled)

16. (canceled)

17. (canceled)

18. (canceled)

19. (canceled)

20. A process for performing an asymmetric hydrogenation reaction which comprises reacting a prochiral or chiral compound in the presence of the catalyst as defined in claim 10 under pressure with hydrogen or a hydrogen source, to produce an optically active compound.

21. (canceled)
